(19) United States
(12) Patent Application Publication
(10) Pub. No.: US 2021/0161447 A1
(43) Pub. Date: Jun. 3, 2021

(54) LANCING DEVICE FOR INFANT WITH A SAFELY ROTATING AND UNLOCKING PUSH-SHOOTING KEY

(71) Applicant: TAICANG MEDLINEER MEDICAL DEVICE TECHNOLOGY CO., LTD., Suzhou City, Jiangsu Province (CN)

(72) Inventor: GANG LI, Suzhou City, Jiangsu Province (CN)

(21) Appl. No.: 16/954,200

(22) PCT Filed: Sep. 10, 2018
(86) PCT No.: PCT/CN2018/104839

§ 371 Date: Jun. 15, 2020

(30) Foreign Application Priority Data
Dec. 15, 2017 (CN) 201711345727.3

(51) Int. Cl. A61B 5/151

(52) U.S. Cl. CPC A61B 5/15113; A61B 2503/04; A61B 5/15126; A61B 5/15142

(57) ABSTRACT

A lancing device for infant with safety rotating and unlocking push-shooting button is provided. The push-shooting button is exposed and occupies two position attitudes. A first attitude is an insurance lock-out state that the push-shooting button is locked to avoid an undesired trigger before actuation, and a second attitude is a state that leaves away from the first one attitude where the push-shooting button is readily loaded to be launched. Under the insurance lock-out state, the push-shooting button is locked and moves only by a rotation. Firstly, the button is rotated and loaded to a to-be-launch state. Then, the button is pressed inwards and an edge lancing mechanism is actuated with a blade projecting from a lancing opening of the shell to cut and retract back into the shell. The whole blood collecting procedure is finished.

### BACKGROUND

### 1. Technical Field

The present invention relates to a medical blood collecting instruments, and more particularly to a cutting device which is usually used to controllably form a precise (or accurate) cutting (or wound/incision) in a patient's body surface. Specifically, it relates to a disposable cutting device which can be economically manufactured, have stable structure and reduce wrongly usage by the users. In this description, the device will be named as a lancing device for infant having a push-shooting button capable of safely rotating and unlocking. This lancing device for infant, which has a push-shooting button capable of safely rotating and unlocking, adopts a cutting type blood collecting method, which is mainly used to collect blood from infant's foot and can also be used to collect blood from other part of a body.

### 2. Description of Related Art

Blood collecting devices or lancing devices are widely used to collect human blood in all kinds of medical units or hospitals, especially used for testing bleeding time or collecting blood sample. More important usage is used to form a cutting on skin of an infant foot and collect blood of the infant for evaluation of metabolic diseases or hereditary diseases. This kind of blood collecting method is suitable for newborns due to a quick movement of the blade.

A great variety of instruments for patient's skin cutting or puncture are sold in the market. The instruments, such as described in U.S. Pat. Nos. 4,643,189, 5,133,730 and Chinese Patent No. 100466973C, are employed to make a cutting on patient's skin with required length and depth.

U.S. Pat. No. 4,643,189 depicts a blood collecting device adopting compression spring to generate actuating energy. When the pre-loaded spring is released, the blade is driven to lance across the patient's skin and a precisely controlled cutting is then formed.

The spring of this kind of device is commonly pre-stretched or pre-compressed in order to accumulate energy to thereby actuate the blade (or make the blade movement) once the blood collecting device is triggered. However, it is harmful to stability of the blood collecting device due to such pre-stretched or pre-compressed. Especially, the pre-stretched or pre-compressed spring may result in undesired early or occasionally triggering or shooting. During assembly of the device, it also perhaps makes the worker suffering from injury because the elements may be occasionally pushed and ricocheted off by such spring. Besides, due to such pre-stretched or pre-compressed actions, other elements relating to the spring will maintain a high internal stress, which in turn results in shorter usage life of the device or poor performance.

It is an important factor to consider the manufacturing cost and/or the assembly cost, since the kind of device is designed for disposable usage. However, it is difficult to assemble the device when the spring is under pre-stretched or pre-compressed states. In some cases, special tools should be employed which result in that the manufacturing cost and the assembly cost are increased. Moreover, insurance structure should be added to prevent the blood collecting device from undesired shooting, such as secured button, blade cover. Obviously, the material cost, the manufacturing cost and the assembly cost are unavoidably increased.

The inventor thoroughly and repeatedly studies the conventional blood collecting devices and finds out the following problems. As for blood collecting devices, as introduced in U.S. Pat. No. 5,133,730, it adopts a method that the high polymer actuating arm is continuedly pressed, then deformed and finally released to thereby generate driven energy. No compressing spring is employed in such device. However, if the user occasionally presses the shooting triggering arm which is in a to-be-shoot state, that is, the shooting triggering arm is pushed inside of a shell, the shooting arm will pivot around and the blade is shooting out. Correspondingly, it will result in useless of the cutting device (this kind of device cannot be used).

In order to avoid undesired early shooting of the device, some products in the market, such as devices introduced in Chinese Patent No. 100466379C, a protecting cap connecting to a blade base is added to the front of the blade with a safety latch formed thereon. The safety latch is used to limit a shooting of the button. Only if the safety latch is removed away, the button can be normally triggered/shot. Although the protecting cap with safety latch resolve the undesired early shooting problem and protect the blade to be sterile and clean, shortcomings still exist. For example, the blade will be damaged when the blade protecting cap is pulled out inclinedly, which in turn increases pains during blood collecting. Further, the protecting cap is so small that easy to lose which will make the management of waste material inconvenience. It will be more dangerous if the protecting cap is picked up by kids who may put such protecting cap into his mouth.

Additionally, it exists defects when employing the continuedly pressing high polymer triggering arm method, in which the arm firstly deforms and then released. During producing stage, if the high polymer triggering arm is not pre-compressed and deformed in advance, the triggering arm has no enough striking force on the blade base when pressing and shooting, which will result in slowly cutting and make the pain increased. If the high polymer triggering arm is pre-compressed and deformed in advance, after a relatively long time of storage, the high polymer will perish which makes the pre-deformation lose the effectiveness and the triggering arm is ageing and curve.

According to the specific laws of some countries, each blood collecting device should be separately sealed up and encased, and the effective date, the standards, the manufacturing number should be all labeled on the outside of package. The steps of sealing up, packaging, sterilization make the blade be more sterile and clean, while the procedure is so complex that the user needs to remove the package first and then pull out the protecting cap, which in turn makes the value of the protecting cap decreased. Nevertheless, the safety latch attached on the protecting cap must be employed to maintain a safety use of the triggering/shooting button. It is desirable to design an improved device with/without blade protecting cap in order not to reduce the elements costs, but also to maintain stability performances.

Hence, based on the conventional technologies, the problems of how to design a low-cost device with long usage life and avoiding undesired shooting and how to design a package method with/without blade protecting cap, are all difficult issues for persons skilled in the art to resolve.

### SUMMARY

The present invention provides a lancing device for infant with a safely rotating and unlocking push-shooting button, which not only employs safety latch structure avoiding undesired shooting in advance, but also employs loaded shooting structure cooperating with the safety latch structure, so that one button structure can accomplish two performances and double effects are achieved step by step. The first objective of the present invention is to resolve problems of assembling difficulty and lifetime attenuation which is resulted from pre-loaded spring, so that, on one hand, the number of components of the device is decreased, on the other hand, the device can be loaded and applied at any time. The second objective of the present invention is to resolve the problem of safety latch.

In order to achieve above-mentioned objectives, the present invention provides a lancing device for infant with safety rotating and unlocking push-shooting button, which includes a shell and actuating lancing system that can be employed as one integral part or configured to consist of an edge lancing mechanism and a triggering and actuating mechanism. The edge lancing mechanism includes a blade and a blade base; or, the edge lancing mechanism is configured to be one integral element/part with a lancing edge. The triggering and actuating mechanism is a push-shooting triggering mechanism which includes a push-shooting button and an actuating arm. The push-shooting button is located on the shell and switches between two positions with respect to the shell by movement therebetween. The triggering and actuating mechanism is employed as one whole part rotatably connecting to the shell. The actuating arm is located inside the shell and the push-shooting button is exposed to outside of the shell opposite to the actuating arm. The one whole part includes a strip wafer connecting thereof and located at an intermediate area, which provides structural elastic for the one whole part. The push-shooting button occupies two kinds of position attitudes during a sliding path with respect to the shell, a first one attitude of which is an insurance lock-out state/position that the push-shooting button is locked to avoid an undesired trigger before actuation, and a second one attitude of which is a state that leaves away from the first one attitude where the push-shooting button is readily loaded to be launched/shot. During lancing, the push-shooting button is firstly locked in the insurance lock-out state/position and then rotated and loaded to a to-be-launch state/position; when the push-shooting button is pressed inwards, the blade base is actuated with the blade projecting from a lancing opening of the shell to make a V-shaped or an arc-shaped cut on a subject's skin and then retracted back into the shell. The whole blood collecting procedure is finished.

Statements and explanations on above-described disclosure are given below.

1. The “shell” refers to a covering structure consisting of two halves of shell interconnected together and provided for the lancing device for infant with safety rotating and unlocking push-shooting button. The “lancing mechanism” refers to a disposable cutting type lancing mechanism. The “insurance lock-out state” refers to an in-movable state when the push-shooting button is under pressing or striking in order to protect the lancing mechanism from occasionally shooting.

2. The actuating lancing system is a name for an assembly of the triggering and actuating mechanism, which is composed by the push-shooting button, the actuating arm and the elastic connecting strip wafer, and the edge lancing mechanism including the blade base and the blade. These functional modules can be assembled as one component/part either partially or totally. The pressing actuating element of the actuating lancing system may employ high polymer material with better elastic, such as nylon, POM, or optimized PC or ABS material. The blade can be made of metal and assembled to the high polymer blade base. Or, the blade and the blade base are integrally made from metal and are hollowed or adopts light metal material to thereby lose the weight. Even, the whole actuating lancing system can be integrally formed by metal.

3. Regarding issues of how to provisionally rotating and positioning of the actuating arm, the following method is provided: there is a provisional positioning structure is formed between the actuating arm and the shell during the rotating route changing from the insurance lock-out state to loaded to-be-launch state with respect to the shell. The provisional positioning structure is composed by a shaft arranged on one part and a shaft hole defined on other part. Since the position can be provisionally confirmed, the strip wafer in the middle of the triggering and actuating mechanism is bent and accumulates elastic energy, during rotating the push-shooting button from the insurance lock-out position to the actuating position.

4. There are two kinds of method for locating and detaching the triggering and actuating mechanism from the shell:

the first kind of method: when pressing the push-shooting button under the shooting state, the releasable pushing block located in a distal end of the push-shooting button couples and squeezes with the actuating arm. There is a circle shaft hole is defined in the actuating arm aimed for provisional rotation. The shaft assembled on the shell has a small shaft height, and the distance from a top end of the shaft to the top of the shell is large. When the actuating arm is coupled and squeezed by the pushing block of the button, the actuating arm will slide and release from the shaft above the top end thereof;

the second kind of method: there is a U-shaped hook provided on the actuating arm and the shaft on the shell has a large shaft height which connects the two opposite side walls of the shell. There is no possibility for the actuating arm detaching from the shaft. When the actuating arm is coupled and squeezed by the pushing block of the button, the actuating arm is bent along the pressing direction until the U-shaped hook released from lateral side of the shaft of the shell.

The advantages and positive effects of the present invention are introduced as follows:

Firstly, the lancing device with the safely rotating and unlocking push-shooting button, not only employs safety locking structure avoiding undesired shooting in advance, but also employs loaded shooting structure cooperating with the safety latch structure, so that the button could work step by step which accomplish two performances and achieve double effects at the same time. The present invention does not need to use elements such as safety button or safety latch worked with the blade protecting cap which may result in undesired launching or shooting. The number of the elements for production is decreased and the costs of production and assembly are reduced. Additionally, the medical staff can use one hand operating at one controlling position to accomplish blood collecting procedure. Compared to conventional technology and conventional device, the present invention adopts one hand operation replacing two hands operation which improves the usage efficiency.

Secondly, the push-shooting button provides, simultaneously, a safely rotating and locking operation together with a loaded to-be-shooting operation. The pre-pressed actuating force is generated. Compared to the conventional similar products, the present invention not only solves the problems on assembling difficulties and lifetime attenuations, but also reduces the usage on the spring components and the purchasing and assembling costs.

### BRIEF DESCRIPTION OF THE DRAWINGS

FIG. 1 is a view showing a push-shooting button of a lancing device according to the present invention under an insurance lock-out state/position.

FIG. 2 is a view showing the push-shooting button of the lancing device according to the present invention along a rotating and unlocking movement route.

FIG. 3 is a view showing the push-shooting button of the lancing device according to the present invention under a loaded to-be-launch state after unlocking.

FIG. 4 is a view showing the lancing device of the present invention under a launched state.

FIG. 5 is a perspective view of FIG. 1.

FIG. 6 is a perspective view of FIG. 3 (showing a vertically guided locking structure).

FIG. 7 is a perspective view of FIG. 4 (showing a locking state after pressing and launching).

FIG. 8 is a partially enlarged view of FIG. 7 (showing a locking state after pressing and launching).

FIG. 9 is a perspective view of a lancing opening of the lancing device according to the present invention.

FIG. 10 is an internal view of the present lancing device showing a circle shaft hole of the actuating arm in accordance with one embodiment—the push-shooting button under an insurance lock-out state with partial shell being removed therefrom.

FIG. 11 is an internal view of the present lancing device showing a circle shaft hole of the actuating arm in accordance with one embodiment—the push-shooting button under the loaded to-be-launch state after unlocking with partial shell being removed therefrom.

FIG. 12 is an internal view of the present lancing device showing a circle shaft hole of the actuating arm in accordance with one embodiment—the push-shooting button under the launched state with partial shell being removed therefrom.

FIG. 13 is a partial perspective view of the present lancing device showing a circle shaft hole of the actuating arm in accordance with one embodiment—the locked actuating arm under the loaded to-be-launch state.

FIG. 14 is a partial perspective view of the present lancing device showing a circle shaft hole of the actuating arm in accordance with one embodiment—the unlocked actuating arm under a shooting state.

FIG. 15 is an internal view of the present lancing device showing a U-shaped hook of the actuating arm in accordance with the other embodiment—the push-shooting button under an insurance lock-out state with partial shell being removed therefrom.

FIG. 16 is an internal view of the present lancing device showing the U-shaped hook of the actuating arm in accordance with the other embodiment—the push-shooting button under the loaded to-be-launch state after unlocking with partial shell being removed therefrom.

FIG. 17 is a partial perspective view of the present lancing device showing the U-shaped hook of the actuating arm in accordance with the other embodiment—the locked actuating arm under the loaded to-be-launch state.

FIG. 18 is a view showing a blood collecting moving route of the blade of the present lancing device with partial shell being removed therefrom.

FIG. 19 is a view showing the present lancing device with a blade protecting hood.

FIG. 20 is an exploded perspective view of the present lancing device.

FIG. 21 is a view showing a triggering and actuating mechanism of the present lancing device.

FIG. 22 is a view showing an edge lancing mechanism of the present lancing device.

FIG. 23 is a view showing the one-piece triggering and actuating lancing system used in the present lancing device.

In these drawings: 1. Shell; 2. Push-shooting button; 3. Actuating arm; 5. Blade base; 6. Rotation shaft; 7. Circle shaft hole; 8. U-shaped hook; 9. Strip wafer; 10. Unlocking guiding slot; 11. Blade edge lancing guiding slot; 12. Actuating guiding post; 13. releasable pushing block; 14. lancing guiding protrusion; 15. actuated striking block; 16. Balance controlling protrusion; 17. anti-ejecting lump; 18. anti-ejecting barb; 19. Shooting-guided slot; 20. Shooting and pressing surface; 21. Lancing opening; 22. Blade-protecting hood.

### DETAILED DESCRIPTION OF THE ILLUSTRATED EMBODIMENT

Reference will now be made to the drawing figures to describe the embodiments of the present disclosure in detail.

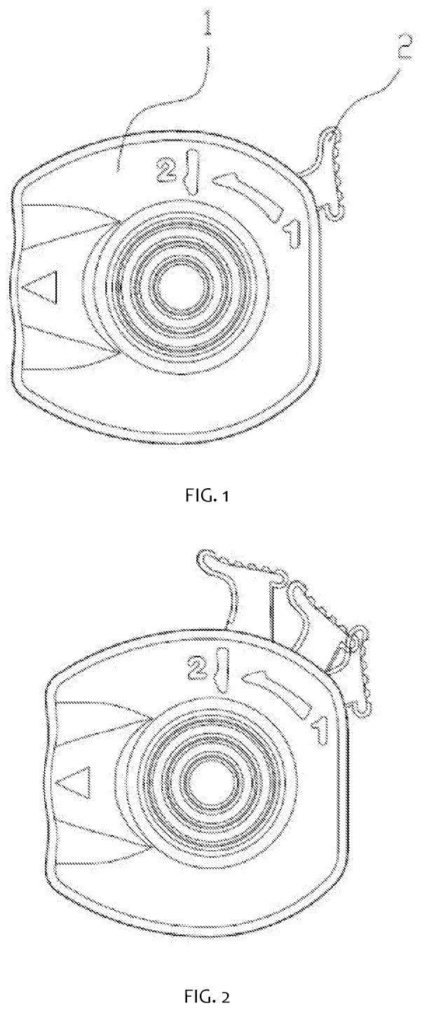

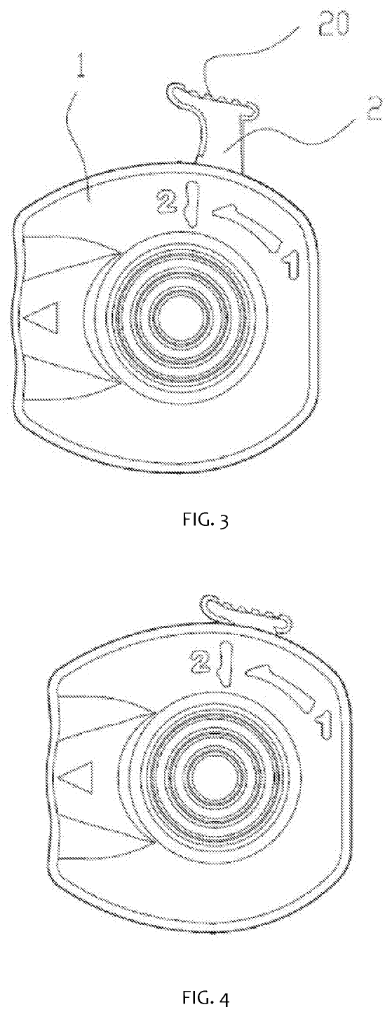

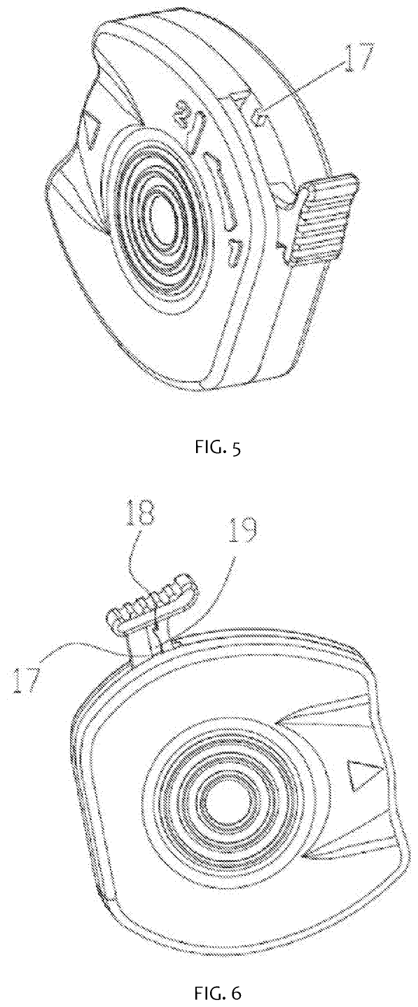

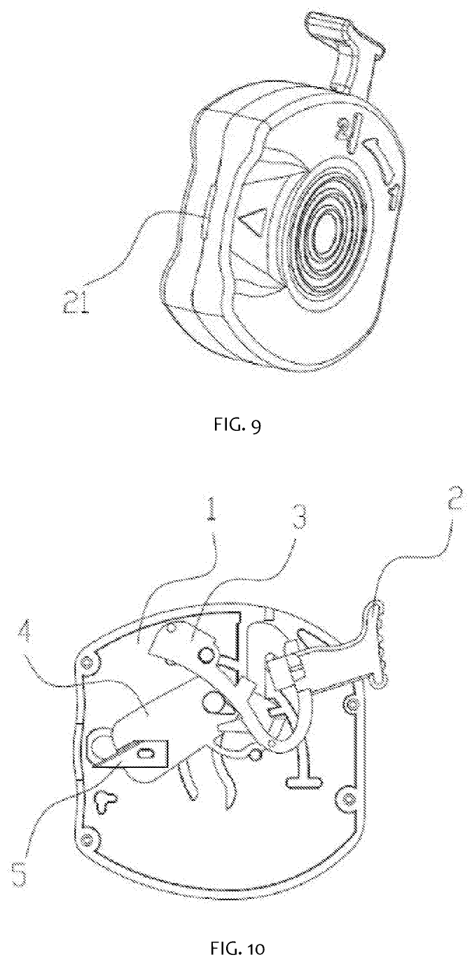

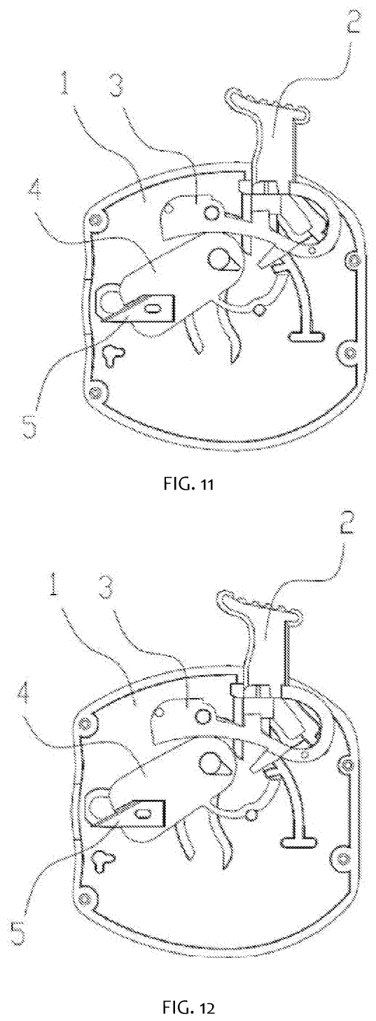

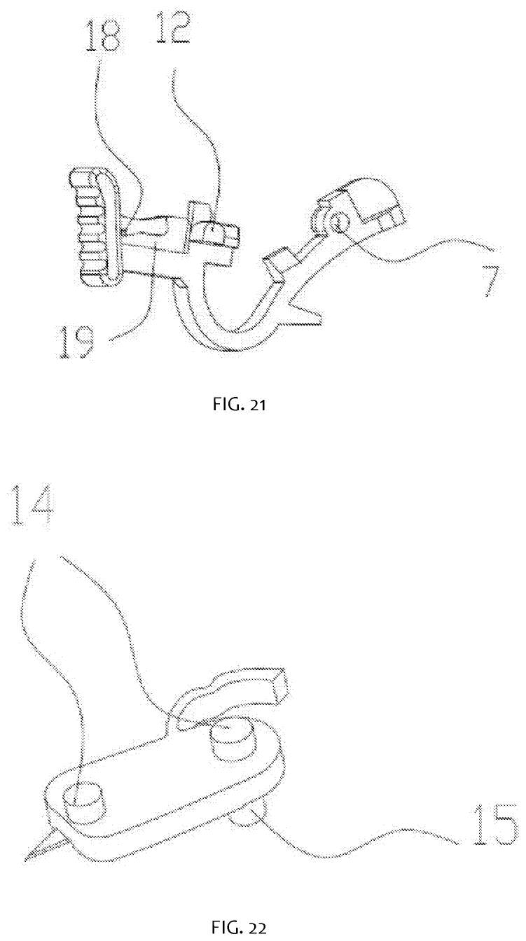

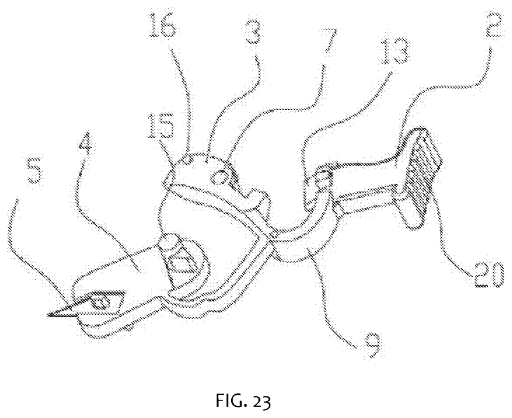

Referring to FIGS. 1 to 23, an illustrated embodiment of the present disclosure discloses a lancing device for infant with safety rotating and unlocking push-shooting button. The lancing device includes a shell 1 and an actuating lancing system that can be employed as one integral part or configured to consist of a triggering and actuating mechanism (FIG. 21) and an edge lancing mechanism (FIG. 22). The edge lancing mechanism includes a blade 4 and a blade base 5. Or, referring to FIG. 23, the edge lancing mechanism is configured to be one integral element/part with a lancing edge. The triggering and actuating mechanism is a push-shooting structure which includes a push-shooting button 2 and an actuating arm 3. The push-shooting button 2 is located on the shell 1 and switches between two positions with respect to the shell 1 by movement therebetween. The triggering and actuating mechanism is employed as one whole part rotatably connecting to the shell. The actuating arm 3 is located inside of the shell 1 and the push-shooting button 2 is exposed to outside of the shell 1 opposite to the actuating arm 3. The push-shooting button 2 occupies two kinds of position attitudes/states during a sliding path/route with respect to the shell 1, a first one attitude of which is an insurance lock-out state/position that the push-shooting button 2 is locked to avoid an undesired trigger before actuation (as shown in FIG. 1 and FIG. 5), and a second one attitude of which is a state that leaves away from the first one attitude where the push-shooting button 2 is readily loaded to be launched/shot (as shown in FIG. 3 and FIG. 6). Under the insurance lock-out state, the push-shooting button 2 is locked at a distal end so that the pressing operation/action cannot make the push-shooting button 2 move/displace. Only if a rotation operation/action is exerted, the push-shooting button 2 can move. During lancing, the push-shooting button 2 is firstly locked in the insurance lock-out state/position and then rotated and loaded to a to-be-launch state/position (as shown in FIG. 2); when the push-shooting button 2 is pressed inwards, the blade base 5 is actuated with the blade 4 projecting from a lancing opening 21 of the shell 1 to make a V-shaped or an arc-shaped cut on a subject's skin and then retracted back into the shell 1. The whole blood collecting procedure is finished.

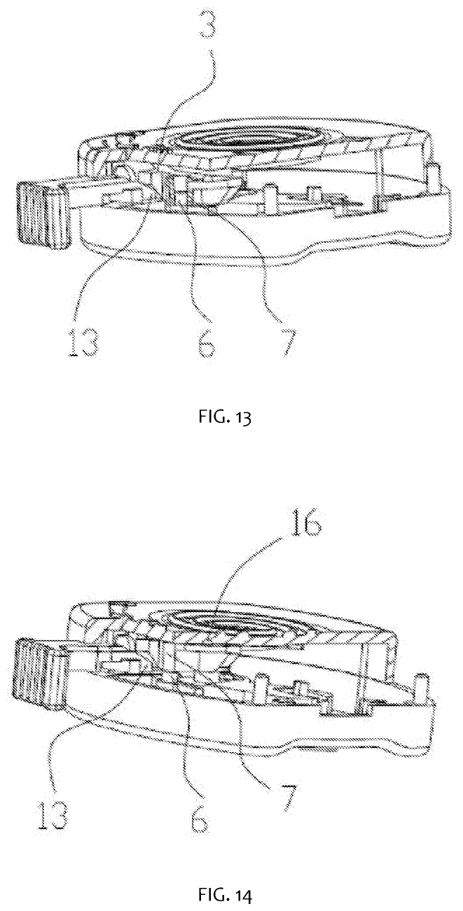

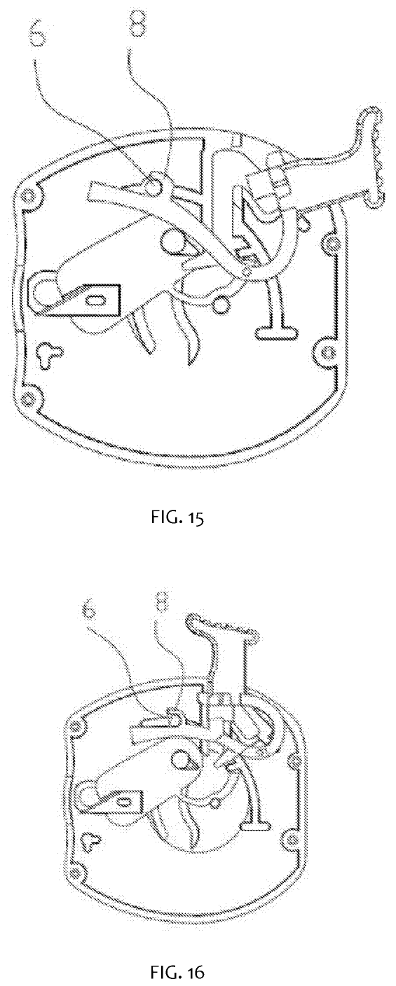

The triggering and actuating mechanism is employed as one whole part/element rotatably connecting to the shell 1 (as shown in FIG. 21). The actuating arm 3 defines a provisional connecting structure thereon and rotatably connects to inside wall of the shell 1 around a rotation shaft 6 through an engagement between the rotation shaft 6 and a shaft hole 7 (as shown in FIG. 10 and FIG. 13). In the other embodiment, the rotation shaft 6 is engageable with a U-shaped hook 8 (as shown in FIG. 15). The push-shooting button 2 has one rigid end exposed outsides from the shell 1. The one whole element includes a strip wafer 9 connecting the push-shooting button 2 with the actuating arm 3. The strip wafer 9 is arranged at an intermediate area of the triggering and actuating mechanism in order to provide structural elastic for the whole element. When the push-shooting button 2 is rotated from the insurance lock-out position to the triggering and launching position, the intermediate strip wafer 9 is deformed and generates elastic potential energy therein due to a distance between the push-shooting button and the actuating arm changing from large to small. The elastic potential energy is released to actuate the edge lancing mechanism movement when the actuating arm 3 is suddenly released at the launching position.

The triggering and actuating mechanism employs an actuating guiding post 12 arranged beside the push-shooting button 2 (as shown in FIG. 21). The actuating guiding post 12 engages with an unlocking guiding slot 10 of the shell 1 to change the states/positions of the push-shooting button 2 along the sliding path thereof (as shown in FIG. 2). A length that the push-shooting button 2 exposed to outside or a distance between the push-shooting button 2 and the shell 3 is changeable because the unlocking guiding slot 10 is such configured that a distance from the unlocking guiding slot 10 to a periphery of the shell 1 is unequal. The length that the push-shooting button 2 exposed to outside is short and is difficult to be touched under the insurance lock-out state, while the length is long under the loaded to-be-launched state which provides enough pressing journey for the button 2 to thereby make the operation/action more comfortable.

According to the disposable lancing device of the present invention, when the push-shooting button 2 of the lancing device of the present invention moves from the first position attitude/state to the second position attitude/state along the sliding path/route with respect to the shell 1, the strip wafer 9 is bent and exerts backwards force onto the button 2 to return to the first position attitude/state. The engagement between the anti-ejecting lump 17 and an anti-ejecting slot or a shooting guiding slot 19, secures the push-shooting button 2 on a loaded to-be-launched state along a vertical direction (as shown in FIG. 6).

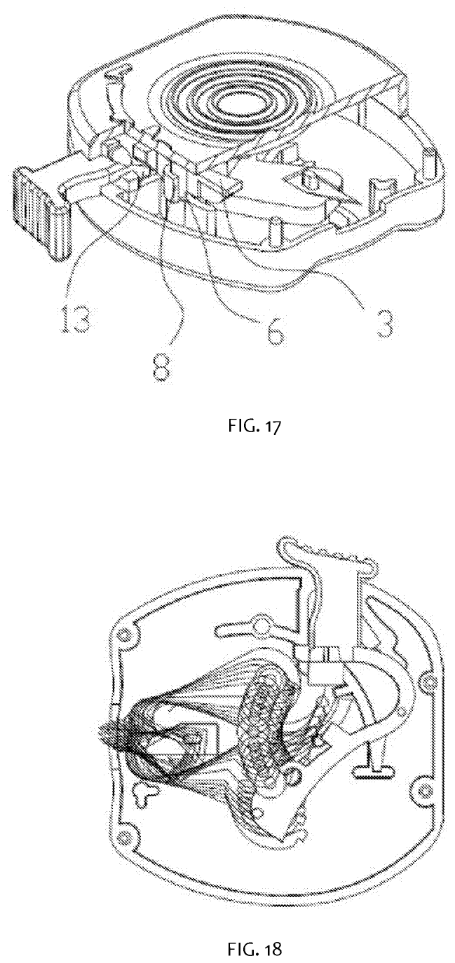

According to the disposable lancing device of the present invention, the push-shooting button 2 of the triggering and actuating mechanism employs a releasable pushing block 13 at a distal end thereof. When the push-shooting button 2 is pressed under the to-be-launch state, the releasable pushing block 13 squeezes and couples with the actuating arm 3 so as to release the circle shaft hole 7 (as shown in FIG. 13) or the U-shaped hook from the rotation shaft 6 and actuating energy is thereby generated. In the one embodiment, the circle shaft hole 7 is provided and employed on the actuating arm 3. The shaft 6 assembled on the shell 1 has a small shaft height, and the distance from a top end of the shaft 6 to the top of the shell 1 is large. When the actuating arm is coupled and squeezed by the pushing block 13 of the button 2, the distal end of the actuating arm 3 is bent and deformed with a balance controlling protrusion 16 formed on the arm 3 subjecting to a vertical force. The force along the pressing direction and the force along a direction vertical to the pressing direction work together in order to drive the shaft hole 7 sliding and finally releasing from the shaft 6 across the top end thereof (as shown in FIG. 14). In the other embodiment, referring to FIG. 17, the U-shaped hook 8 is provided and employed on the actuating arm 3. The shaft 6 on the shell 1 has a large shaft height which connects two opposite side walls of the shell 1. When the actuating arm 3 is coupled and squeezed by the pushing block 13 of the button 2, the actuating arm 3 is bent along the pressing direction until the U-shaped hook 8 released from lateral side of the shaft 6 of the shell 1.

The blade edge lancing mechanism comprises a pair of lancing guiding protrusions 14 and an actuated striking block 15 (as shown in FIG. 20). The pair of lancing guiding protrusions 14 are engageable with the pair of blade edge lancing guiding slots 11, respectively. One of the blade edge lancing guiding slots 11 is defined to have a shape approximate to a curve and the other one of the blade edge lancing guiding slots is defined to have a shape approximate to a straight line. When the actuating arm 3 of the triggering and actuating mechanism is released, the elastic potential energy makes the actuating arm 3 push the actuated striking block 15 of the blade edge lancing mechanism to move. One of the lancing guiding protrusions 14 moves along a curved path in the curved guiding slot 11, and the other one of the lancing guiding protrusions 14 moves forwards and then backwards in the straight line shape guiding slot 11. As can be understood, the rotating movement of the actuating arm 3 turns into a rotation and a translation of the blade 4. Consequently, the blade 4 makes an arced or V-shaped cutting and then retracts back into the shell 1. The blood collecting procedure is finished.

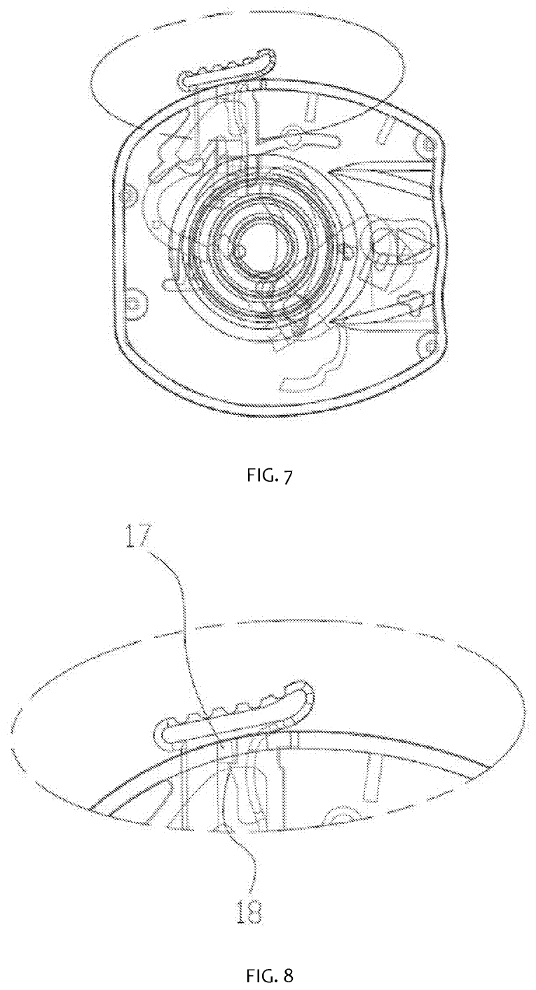

The push-shooting button 2 has an anti-ejecting barb 18 formed thereon. The shell 1 has an anti-ejecting lump 17 formed thereon for engaging and locking with the anti-ejecting barb 18. When the launching action of the push-shooting button 2 is accomplished, the barb 18 and the lump 17 interlocks with each other to thereby prevent the push-shooting button 2 back to an initial to-be-shoot state/position (as shown in FIGS. 7-8). At this situation, the blade 4 is restricted and stopped by the front end of the actuating arm 3 (as shown in FIG. 12). The blade is unable to move so as to avoid the blade bringing therewith blood from cutting the body which will result in second time damage and crossover infection. Moreover, it is convenient for the user to recognize the status of the button 2 after pressing and locking the button 2.

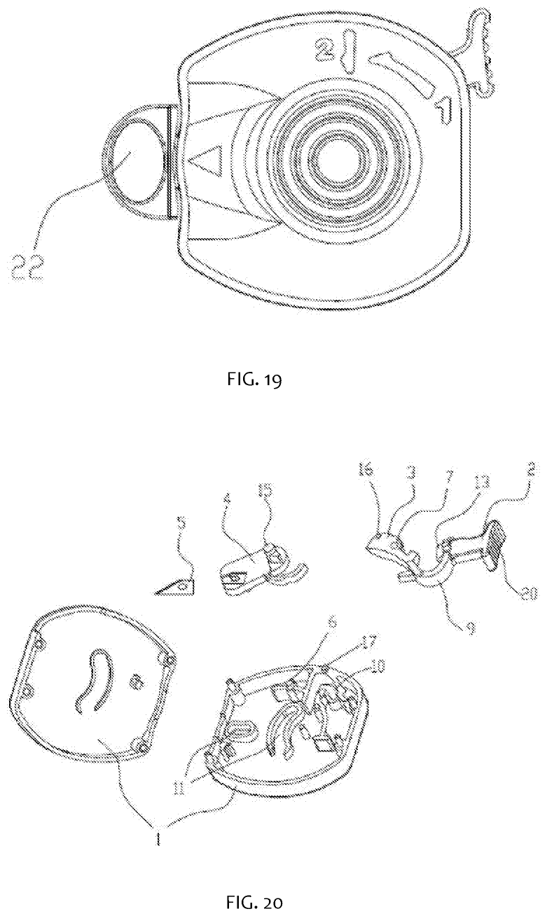

A blade-protecting hood 22 is selectively provided, which can be tore open or snapped off to thereby detach from the blade 4 (as shown in FIG. 19). The blade-protecting hood 22 helps to maintain the sharpness of the blade 4 and make the blade 4 sterile and clean. The employment of such blade protecting hood 22 enable the lancing device to satisfy rules or habits of some countries.

1. A lancing device for infant with an actuating button adapted for safely rotating and unlocking, comprising a shell and an actuating lancing system;
said actuating lancing system comprising a triggering and actuating mechanism and an edge lancing mechanism;
said edge lancing mechanism having a blade and a blade base; or, said edge lancing mechanism formed as one integral element with lancing edge;
said triggering and actuating mechanism is a pushing actuating-type structure, including a push-shooting button and an actuating arm, said push-shooting button slidably located on said shell and switching between two positions with respect to said shell by movement therebetween;
wherein said push-shooting button is exposed to outside of said shell, said push-shooting button occupies two kinds of position attitudes during a sliding path with respect to said shell; a first one attitude is an insurance lock-out state/position that said push-shooting button is locked to avoid an undesired trigger before actuation, and a second one attitude is a state that leaves away from the first one attitude where said push-shooting button is readily loaded to-be-launched/shot; during lancing, said push-shooting button is firstly locked in the insurance lock-out state/position and then rotated and loaded to a to-be-launch state/position; when the push-shooting button is pressed inwards, said blade base is actuated with said blade projecting from a lancing opening of said shell to make a V-shaped or an arc-shaped cut on a subject's skin and then retracted back into said shell.

2. The lancing device as claimed in claim 1, wherein said triggering and actuating mechanism works as one integral component which is rotatably connecting to said shell, said one integral component has an actuating arm formed therein, said actuating arm defines a provisional connecting structure thereon and rotatably connects to inside wall of said shell around a rotation shaft through an engagement between said rotation shaft and a shaft hole or an engagement between said rotation shaft and a U-shaped hook; wherein said push-shooting button has one rigid end exposed outsides from said shell, and said one integral component includes a strip wafer connecting thereof and located at an intermediate area thereof in order to provide structural elastic for said one integral component; wherein when said push-shooting button is rotated from said insurance lock-out position to said triggering and launching position, said intermediate strip wafer, connecting said push-shooting button with said actuating arm, is deformed and generates elastic potential energy therein due to a distance between said push-shooting button and said actuating arm changing from large to small; and wherein said elastic potential energy is released to actuate said edge lancing mechanism movement when said actuating arm is suddenly released at said launching position.

3. The lancing device as claimed in claim 1, wherein said triggering and actuating mechanism and said edge lancing mechanism of said actuating lancing system are either worked as one whole part or worked as two separated elements.

4. The lancing device as claimed in claim 1, wherein said shell has an inside wall defining an unlocking guiding slot used for said actuating button and a pair of blade edge lancing guiding slot; wherein said unlocking guiding slot is adapted to guide said actuating button slidable moving therein and switching between said insurance lock-out state/position and said to-be-launch state/position; and wherein said blade edge lancing guiding slot leads said blade edge lancing mechanism moving therealong so as to make said blade extend outward from said lancing opening of said shell and generate a V-shaped or an arc-shaped cut.

5. The lancing device as claimed in claim 4, wherein said triggering and actuating mechanism employs an actuating guiding post arranged beside said push-shooting button, which engages with said unlocking guiding slot of said shell to change said states/positions of said push-shooting button along said sliding path; and wherein a length that said push-shooting button exposed to outside or a distance between said push-shooting button and said shell is changeable because said unlocking guiding slot is such configured that a distance from said unlocking guiding slot to a periphery of said shell is unequal; and wherein said length that said push-shooting button exposed to outside is short and difficult to be touched during said insurance lock-out state, while said length is long during said to-be-launched state.

6. The lancing device as claimed in claim 1, wherein said push-shooting button of said triggering and actuating mechanism employs a releasable pushing block at a distal end thereof; wherein when said push-shooting button is pressed in said to-be-launch state, said releasable pushing block squeezes and couples with said actuating arm so as to release said shaft hole or said U-shaped hook from said rotation shaft and actuating energy is thereby generated.

7. The lancing device as claimed in claim 6, wherein said blade edge lancing mechanism comprises a pair of lancing guiding protrusions and an actuated striking block, said pair of lancing guiding protrusions engaging with said pair of blade edge lancing guiding slots, respectively; wherein one of said blade edge lancing guiding slots is defined to have a shape approximate to a curve and the other one of said blade edge lancing guiding slots is defined to have a shape approximate to a straight line; and wherein when said actuating arm of said triggering and actuating mechanism is released, said elastic potential energy makes said actuating arm push said actuated striking block of said blade edge lancing mechanism to move, and subsequently, said pair of lancing guiding protrusions are driven to engage and move in corresponding blade edge lancing guiding slots, so that a rotation and a translation of said blade are fulfilled and finished.

8. The lancing device as claimed in claim 1, wherein said push-shooting button has an anti-ejecting barb formed thereon and said shell has an anti-ejecting lump formed thereon for engaging and locking with said anti-ejecting barb, when said launching action of said push-shooting button is accomplished.

9. The lancing device as claimed in claim 1, further comprising a blade-protecting hood, which can be tore open or snapped off to thereby detach from said blade.
